(19) United States
(12) Patent Application Publication
(10) Pub. No.: US 2016/0166176 A1
(43) Pub. Date: Jun. 16, 2016

(54) COMPUTER-BASED ANALYSIS OF OSCILLATORY VENTILATION

(71) Applicant: Mayo Foundation for Medical Education and Research, Rochester, MN (US)

(72) Inventors: Bruce D. Johnson, Rochester, MN (US); Thomas P. Olson, Cannon Falls, MN (US)

(21) Appl. No.: 14/907,364

(22) PCT Filed: Jul. 23, 2014
(86) PCT No.: PCT/US14/47890

§ 371 Date: Jan. 25, 2016

Related U.S. Application Data
(60) Provisional application No. 61/858,487, filed on Jul. 25, 2013.

(51) Int. Cl. A61B 5/091; A61B 5/087; A61B 5/00

(52) U.S. Cl. CPC A61B 5/091; A61B 5/7257; A61B 5/7275; A61B 5/087

(57) ABSTRACT

This document provides systems and techniques that can be used to provide computer-implemented interpretation (e.g., up-to-date, on demand, and real time interpretation) of respiratory profiles of patients using, for example, an application provided by a computer server system, to assess a patient's cardiac and/or pulmonary function.

### CROSS-REFERENCE TO RELATED APPLICATIONS

This application claims the benefit of U.S. Provisional Application Ser. No. 61/858,487, filed Jul. 25, 2013. The disclosure of the prior application is considered part of (and is incorporated by reference in) the disclosure of this application.

### BACKGROUND

1. Technical Field

This document relates to computer-implemented interpretation (e.g., up-to-date, on demand, and real time interpretation) of respiratory profiles of patients using, for example, an application provided by a computer server system, to assess a patient's cardiac and/or pulmonary function.

2. Background Information

With the advent of new diagnostic technologies, improved survival of patients, and the increasing age of the population, the prevalence of chronic heart failure (HF) has increased and the incidence is expected to remain high over the next several decades. The pathophysiology of this syndrome includes classic systolic left ventricular dysfunction and the more recently recognized HF with preserved ejection fraction; both of which can be complicated by altered vascular, metabolic, endocrine, neurologic, and pulmonary physiology. Cardiopulmonary exercise testing (CPET) with spirometry and gas exchange analysis can provide information about the integrative nature of these systems, disease severity, and prognosis in HF patients.

### SUMMARY

This document provides systems and techniques that can be used to provide computer-implemented interpretation (e.g., up-to-date, on demand, and real time interpretation) of respiratory profiles of patients using, for example, an application provided by a computer server system, to assess a patient's cardiac and/or pulmonary function. For example, this document provides methods and materials for computer-based analysis of oscillatory ventilation for tracking health status and/o predicting risk of decompensation in patients with heart failure.

As described herein, rapid quantitation of spirometry and gas exchange values can be obtained at rest and during exercise and used to identify quantifiable cyclic patterns in breath-by-breath ventilation and gas exchange and to define cycle amplitude and frequency as well as other information embedded in breathing patterns that are not typically reported from spirometry or with cardiopulmonary exercise testing. For example, the calculation and modeling techniques described herein can be used to analyze resting PB (periodic breathing), OB (oscillatory breathing), and/or EOV (exercise oscillatory ventilation) patterns embedded within the typical commercial spirometry systems or gas exchange data output against hand scored values of ventilation (\dot{V}_{E}), tidal volume (V_{T}), oxygen uptake (\dot{V}O₂), and partial pressure of end-tidal CO₂ (P_{ET}CO₂). In some cases, amplitudes from a peak detection method and periods from either a Fourier method or sine wave method can be used to determine breathing patterns.

In one aspect, this document features a computer-implemented method. The method comprises, or consists essentially of, (a) accessing, by a computer system, cardiopulmonary data for a patient; (b) identifying, using an oscillation period operation, periods of oscillation from the cardiopulmonary data for the patient; (c) identifying, using peak detection operation, amplitudes of oscillation from the cardiopulmonary data for the patient; (d) determining, by the computer system, one or more breathing patterns for the patient based on (i) the identified periods of oscillation and (ii) the identified amplitudes of oscillation; and (e) outputting, by the computer system, information that identifies the one or more breathing patterns. The oscillation period operation can comprise a sine wave fitting operation that identifies the periods of oscillation. The oscillation period operation can comprise Fourier analysis operation that identifies the periods of oscillation. The cardiopulmonary data for the patient can correspond to measurements that were taken over a period of time while the patient was exercising. The cardiopulmonary data for the patient can correspond to measurements that were taken over a period of time while the patient was resting. The cardiopulmonary data can comprise spirometry and gas exchange values for the patient. The one or more breathing patterns can comprise cyclic patterns in breath-by-breath ventilation and gas exchange. The determining can comprise curve fitting the cardiopulmonary data for the patient by combining the periods of oscillation that are identified using the oscillation period operation with the amplitudes of oscillation that are identified using the peak detection operation. The peak detection operation can comprise (a) for each data point in the cardiopulmonary data, generating a list that includes at least a threshold number of adjacent data points that occur earlier and later in time within the cardiopulmonary data; (b) sorting the generated list; (c) discarding data points in the generated list have the greatest and lowest values to generate a resulting list; (d) identifying local minima and maxima from the resulting list; (e) determining time differences and magnitude differences between the local minima and maxima; and (f) averaging the time differences and the magnitude differences to determine magnitude and period values from the cardiopulmonary data.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Representative example of periodic breathing at rest and exercise oscillatory ventilation in one HF patient.

FIG. 2. (Left Column) Linear regression analysis of measurement techniques compared to manual hand scoring for the amplitude of oscillations in \dot{V}_{E} during resting periodic breathing for: A1) sine wave algorithm and A2) peak detection algorithm. (Right Column) Linear regression analysis of measurement techniques compared to manual hand scoring for the amplitude of oscillations in \dot{V}_{E} during exercise oscillatory ventilation for: B1) sine wave algorithm and B2) peak detection algorithm.—=line of identity. - - - =line of best fit.

FIG. 3. (Left Column) Linear regression analysis of measurement techniques compared to manual hand scoring for the period of oscillations in \dot{V}_{E} during resting periodic breathing for: A1) sine wave algorithm, A2) peak detection algorithm, and A3) Fourier analysis. (Right Column) Linear regression analysis of measurement techniques compared to manual hand scoring for the period of oscillations in \dot{V}_{E} during exercise oscillatory ventilation for: B1) sine wave algorithm, B2) peak detection algorithm, and B3) Fourier analysis.—=line of identity. - - - =line of best fit.

FIG. 4. Fourier analysis for measurement of the frequency content of breathing cycles. This analysis quantifies the width of the peaks and identifies secondary peaks in the frequency domain. A) Example of a discrete frequency peak, a pattern easily fit with a best-fit sine wave. B) Example of a larger spread in frequency components demonstrating the complexity of fitting a single-frequency sine wave.

FIG. 5. Block diagram of computing devices that may be used to implement the systems and methods described in this document, as either a client or as a server or plurality of servers.

### DETAILED DESCRIPTION

This document provides systems and techniques that can be used to provide computer-implemented interpretation (e.g., up-to-date, on demand, and real time interpretation) of respiratory profiles of patients using, for example, an application provided by a computer server system, to assess a patient's cardiac and/or pulmonary function. For example, this document provides algorithms that can be used to quantify cyclic patterns rapidly in breath-by-breath ventilation and gas exchange data. One example of an algorithm that can be used to determine the amplitude is a peak detection method provided herein. Examples of algorithms that can be used to determine the period are the Fourier method and sine wave method provided herein.

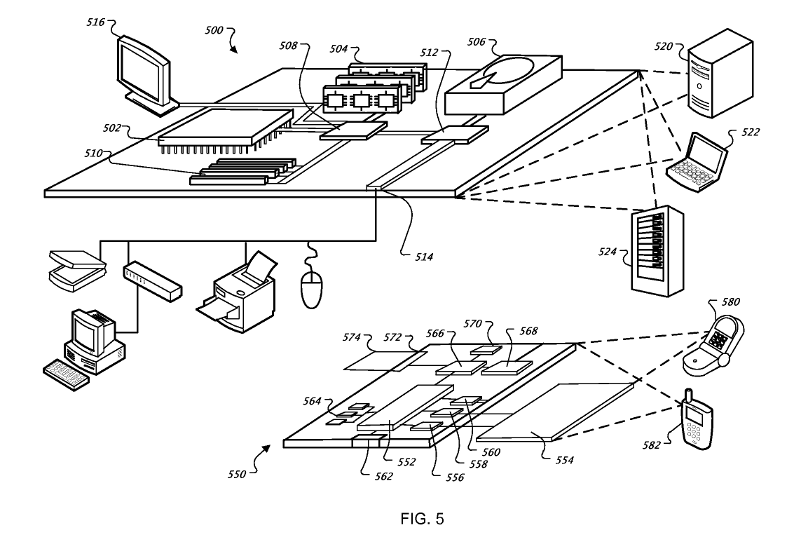

As described below with regard to FIG. 5, the algorithms, techniques, and systems that are described in this document can be implemented using any of a variety of appropriate computing devices (e.g., laptop computer, desktop computer, smartphone, tablet computing device, medical device) and/or computer systems (e.g., two or more computing devices communicating over one or more communication channels, computer server system). For example, a patient monitoring computing device may be programmed to perform one or more of the algorithms and techniques that are described in this document. Additionally, the algorithms, techniques, and systems may be implemented in any of a variety of ways, such as through software, firmware, and/or hardware (e.g., application specific integrated circuit (ASIC)).

In some cases, the algorithms, techniques, and systems provided herein can be used with exercise and/or with applications other than exercise. For example, the algorithms, techniques, and systems provided herein can be integrated with measurements of breathing rate and/or depth to provide an index of disease severity, a likelihood of congestion or decompensation, and/or a need for more aggressive treatment. In some cases, the algorithms, techniques, and systems provided herein can be used with exercise systems (e.g., the Shape system), medical graphics systems, carefusion, and other systems. The likelihood of death increases when oscillatory patterns are detected, and the disease process could be characterized as follows: stable heart failure→worsening disease→developing pulmonary congestion→rising left heart pressure→chaotic breathing→oscillatory or PB→decompensation→weight increases→decompensation and hospitalization. The methods and materials provided herein can allow a physician to identify and treat in an accurate and rapid manner those patients suspected to progress to decompensation, weight increases, decompensation, and hospitalization.

The invention will be further described in the following examples, which do not limit the scope of the invention described in the claims.

### EXAMPLES

### Example 1

### Quantifying Oscillatory Ventilation during Exercise in Patients with Heart Failure

### Population Characteristics

Eleven adult male patients from the Mayo Clinic Heart Failure Service undergoing clinically indicated right heart catheterization for potential cardiac transplantation and who had previously demonstrated clear periodic breathing at rest and during exercise volunteered for the study.

### Exercise Testing

All participants were asked to avoid strenuous physical activity for 24 hours and refrain from eating or consuming non-clear liquids for 8 hours prior to arrival to the cardiac catheterization laboratory at 6:00 am. While supine, prior to clinical right sided cardiac catheterization, participants underwent 10 minutes of resting ventilation and gas exchange data collection. After resting data collection and clinical procedure data collection (˜25-30 minutes of procedural time), participants remained supine and underwent a maximal exercise test using a supine cycle ergometer (American Echo, Inc.. Kansas City, Mo.). During this exercise test, participants were verbally encouraged to continue the exercise to maximal exertion, identified as a rating of perceived exertion (RPE)≧17 on the Borg 6-10 scale or a respiratory exchange ratio (RER) of ≧1.0.

### Measurement of Ventilation and Gas Exchange

Oxygen consumption (\dot{V}O₂), Ventilation (\dot{V}_{E}), Tidal Volume (V_{T}), and partial pressure of end-tidal carbon dioxide (P_{ET}CO₂) were measured with a metabolic measurement system (MedGraphics CPX/D; Medical Graphics, St. Paul, Minn.) utilizing a mouth piece, pneumotach, gas analyzers, and nose clip at rest and during supine cycle ergometry. Manual volume calibration was performed with a three liter syringe, whereas gas calibration was performed with gases of known concentration. Calibration was performed immediately prior to each procedure.

### Definition and Manual Measurement of PB and EOV

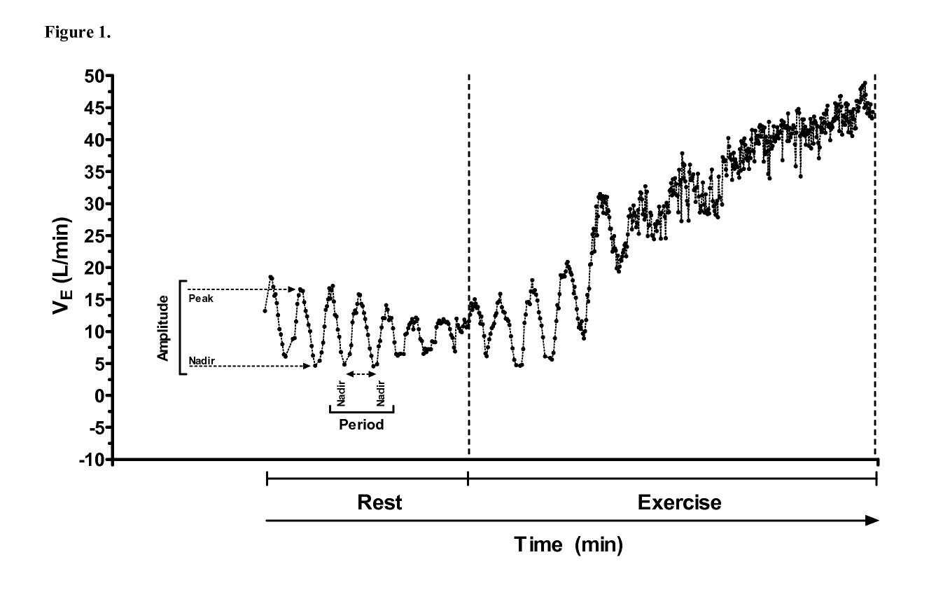

For purposes of identifying study participants, PB and EOV were defined as 1) clear regular cyclic waxing and waning of \dot{V}_{E} without apnea, amplitude of \dot{V}_{E}>30% of mean \dot{V}_{E} at cycle peak, and a minimum of 3 consecutive cycles at rest and during exercise as described elsewhere and shown in FIG. 1 (Olson et al., Chest, 133:474-481 (2008); Olson et al., Am. Heart J., 153:e1-e7 (2007); and Ponikowski et al., Circulation, 100:2418-2424 (1999)). Hand scoring of \dot{V}_{E}, V_{T}, P_{ET}CO₂, and \dot{V}O₂ for both PB and EOV were completed using breath-by-breath data imported from a metabolic measurement system (MedGraphics CPX/D: Medical Graphics, St. Paul, Minn.) to Microsoft Excel (Microsoft Corporation, Redmond, Wash.). Figures of \dot{V}_{E}, V_{T}, P_{ET}CO₂, and \dot{V}O₂ (y-axis) over time (x-axis) were then generated for manual measurement of cycle amplitude and period. Amplitude was measured as the difference between peak and nadir of a cycle and averaged over three consecutive cycles. Period was measured as the time interval between two consecutive nadirs or two consecutive peaks and averaged over three consecutive cycles. The same three cycles were used for the measurement of amplitude and period.

### Algorithm Detection and Measurement of PB and EOV

### Peak Detection Analysis

The first method was designed to reproduce the method used by a human observer during manual data analysis. The same breath-by-breath data used in the manual scoring for \dot{V}_{E}, V_{T}, P_{ET}^{CO}₂, and \dot{V}O₂ were first smoothed by picking the best-5-of-7 at every point as follows. At each data point, a temporary list of the current breath plus the previous and the later three breaths was constructed and sorted. The highest and lowest points were discarded, and a mean of the remaining five were used. A peak detection algorithm then located the local minima and maxima. The time differences and magnitude differences of pairs of maxima and minima were then averaged to determine magnitude and period, respectively.

### Sine Wave Analysis

The second method again used breath-by-breath data for \dot{V}_{E}, V_{T}, P_{ET}CO₂, and \dot{V}O₂ over the same period used for manual analysis. A sine wave model was fit to the data:

Y=Int+Slope×Time+MAG×sin(2×PI×(t−t0)/T+Phase) (Eq.1)

In Eq. 1, Int and Slope are linear intercept and slope terms to account for linear increases in the variable of interest with time, MAG is the magnitude of the sine wave component, T is the period, and Phase is the phase shift of the component. The variables ‘t’ and ‘t0’ refer to breath-by-breath time, and start time of the analyzed breaths, respectively. Phase shifts were arbitrarily expressed relative to the phase determined for \dot{V}_{E}. Data for \dot{V}_{E} were first fit to the sine wave model allowing all five parameters to be fit (Int, Slope, MAG, T, and Phase). For all other variables, T was fixed at the value found for \dot{V}_{E}, and only four parameters were fit. Powell minimization (Press et al., Numerical Recipes: The art of scientific computing. Cambridge University Press, 2007) was used to optimize the fit by minimizing the sum of squared differences between the sine wave model output and the original data. Statistical significance of the modeling was determined from partial F-test analysis. In this way, the simple linear model (Y=Int+Slope×Time) can be compared with the addition of the sine wave components. In the cases analyzed herein, the sine wave components were all statistically significant at p<0.05, though in future studies the goodness of fit can be used to determine significant sine wave components.

### Fourier Analysis

Fourier analysis was applied to the same breath-by-breath data analyzed by hand. Data were first re-sampled using simple linear interpolation against time to create data streams in even one-second intervals. Linear shifts in the data with time were first removed by subtracting the linear component determined from the sine wave analysis, above. The data were then shifted vertically by the mean, and finally a zero-padding region was added to the beginning and end of the data. Zero pad regions were at least as large as the original data stream. The pre-processed data were submitted to fast Fourier analysis (Press et al., Numerical Recipes: The art of scientific computing. Cambridge University Press, 2007). The peaks in the frequency domain were sorted by magnitude, and the main frequency peak identified. Lesser peaks were also retained, but not analyzed. The period represented by the main frequency peak was determined from 1/F.

For all analysis methods (manual and algorithm), the mean of each variable was also reported by taking the simple average of all data points in the interval analyzed.

Statistical Analysis Statistical analysis and graphic presentation were accomplished using SPSS (v 12.0, Chicago, Ill.) and GraphPad Prism (v 4.0, San Diego, Calif.), respectively. Two-tailed paired t-tests were used to compare ventilation and gas exchange variables between the hand and algorithm measurements. Pearson's correlation coefficients were calculated between measurement techniques. For graphical purposes, the primary focus for the correlation figures was placed on the measures of \dot{V}_{E}. Statistical significance was set at an alpha level of 0.05. All data were presented as mean±standard deviation (SD).

### Results

### Participant Characteristics

Patient characteristics and medication usage is shown in Table 1. These patients presented as slightly overweight based on BMI with a New York Heart Association (NYHA) Class range of III-IV. The mean ejection fraction (EF) of 17.3±4.0%, and the peak VO₂ of 9.2±2.6 mL/kg/min were both severely reduced.

TABLE 1
Participant characteristics.

| | Mean ± SD | Range |
|---|---|---|
| Demographics (n = 11) | | |
| Age (yr) | 53 ± 8 | 45-71 |
| Height (cm) | 175.1 ± 7.1 | 165-187 |
| Weight (kg) | 81.9 ± 15.1 | 65-114 |
| BMI (kg/m²) | 26.7 ± 4.5 | 21-37 |
| Clinical Characteristics | | |
| EF (%) | 17.3 ± 4.0 | 8-22 |
| E/e′ (n = 9) | 27.4 ± 11.1 | 10-40 |
| LV mass index (gm/m²) | 158.3 ± 52.3 | 114-272 |
| Peak \dot{V}O₂ (mL/kg/min) | 9.2 ± 2.6 | 6-13 |
| NYHA class | Class III = 7 | |
| | Class IV = 4 | |
| Medications | | |
| Digoxin | 9 (82%) | |
| ACE | 5 (45%) | |
| Diuretics | 10 (91%) | |
| β-Blockers | 5 (45%) | |
| ARB | 4 (36%) | |

BMI, body mass index; EF, ejection fraction; E, early diastolic mitral inflow velocity; e′; early diastolic mitral annular velocity; LV, left ventricle; \dot{V}O₂, oxygen consumption; NYHA, New York Heart Association; ACE, angiotensin converting enzyme; ARB, angiotensin receptor blocker. Data are reported as mean ± SD, range, or number (% of population) as appropriate.

### Resting Periodic Breathing

Table 2 details the mean (absolute), amplitude, and period for \dot{V}_{E}, V_{T}, P_{ET}CO₂, and \dot{V}O₂ at rest during PB. There was no difference between the manual hand score and the sine wave algorithm for mean absolute values of \dot{V}_{E}, V_{T}, P_{ET}CO or \dot{V}O₂. Values for peak detect and Fourier methods were the same as for sine fit, and were not listed in the table. Similarly, there were no differences between the hand score and the peak detection algorithm for amplitude for the same variables. The sine wave fit tended to produce smaller amplitude estimates compared to hand score or the peak detect method, and these differences were significant for P_{ET}CO₂, and VO₂. The peak detect method produced shorter periods compared to manual (P<0.05 for all variables). The periods for the sine fit method were the same among the variables. For all variables other than the amplitude of V_{T}, the correlation coefficients between the manual hand score and peak detect algorithm (Table 3) appeared to be lower compared to the correlations between the manual hand score and sine wave fit (Table 3, not tested statistically).

TABLE 2
Analysis of Periodic Breathing at rest across Measurement Techniques.

| | Manual | Peak Detect | Sine Fit | Fourier |
|---|---|---|---|---|
| Mean (Absolute) | | | | |
| \dot{V}_{E} (L*min⁻¹) | 9.98 ± 1.88 | — | 9.36 ± 2.01 | — |
| V_{T} (L) | 0.60 ± 0.28 | — | 0.57 ± 0.29 | — |
| P_{ET}CO₂ (mmHg) | 32.5 ± 3.4 | — | 32.5 ± 3.4 | — |
| \dot{V}O₂ (L*min⁻¹) | 0.26 ± 0.06 | — | 0.23 ± 0.05 | — |
| Amplitude | | | | |
| \dot{V}_{E} (L*min⁻¹) | 9.54 ± 4.27 | 9.91 ± 4.75 | 8.63 ± 4.41 | — |
| V_{T} (L) | 0.55 ± 0.37 | 0.58 ± .40 | 0.45 ± .30 | — |
| P_{ET}CO₂ (mmHg) | 7.27 ± 3.72 | 6.61 ± 4.50 | 6.13 ± 3.49 * | — |
| \dot{V}O₂ (L*min⁻¹) | 0.29 ± 0.16 | 0.31 ± 0.19 | 0.24 ± 0.14 * | — |
| Period (minutes) | | | | |
| \dot{V}_{E} | 1.11 ± 0.43 | 0.77 ± 0.14 * | 1.11 ± 0.44 | 1.10 ± 0.43 |
| V_{T} | 1.06 ± 0.42 | 0.65 ± .21 * | 1.11 ± 0.44 | 1.04 ± 0.47 |
| P_{ET}CO₂ | 1.15 ± 0.54 | 0.83 ± 0.44 * | 1.11 ± 0.44 | 1.08 ± 0.41 |
| \dot{V}O₂ | 1.08 ± 0.41 | 0.71 ± 0.21 * | 1.11 ± 0.44 | 1.08 ± 0.44 |

\dot{V}_{E}, minute ventilation; V_{T}, tidal volume; P_{ET}CO₂, partial pressure of end tidal carbon dioxide; \dot{V}O₂, oxygen uptake. Data are reported as mean ± SD. All comparisons vs. Manual.
* = P < 0.05

TABLE 3
Pearson Correlation Analysis of Periodic Breathing at rest.

| | Peak Detect | Sine Fit | Fourier |
|---|---|---|---|
| Mean (Absolute) | | | |
| \dot{V}_{E} | — | 0.81 (0.003) | — |
| V_{T} | — | 0.96 (<0.001) | — |
| P_{ET}CO₂ | — | 0.99 (<0.001) | — |
| \dot{V}O₂ | — | 0.66 (0.027) | — |
| Amplitude | | | |
| \dot{V}_{E} | 0.90 (<0.001) | 0.94 (<0.001) | — |
| V_{T} | 0.94 (<0.001) | 0.894 (<0.001) | — |
| P_{ET}CO₂ | 0.89 (<0.001) | 0.966 (<0.001) | — |
| \dot{V}O₂ | 0.91 (<0.001) | 0.952 (<0.001) | — |
| Period (minutes) | | | |
| \dot{V}_{E} | 0.59 (0.058) | 0.99 (<0.001) | 0.98 (<0.001) |
| V_{T} | 0.80 (0.003) | 0.98 (<0.001) | 0.98 (<0.001) |
| P_{ET}CO₂ | 0.57 (0.068) | 0.90 (<0.001) | 0.78 (0.004) |
| \dot{V}O₂ | 0.64 (0.035) | 0.97 (<0.001) | 0.97 (<0.001) |

\dot{V}_{E}, minute ventilation; V_{T}, tidal volume; P_{ET}CO₂, partial pressure of end tidal carbon dioxide; \dot{V}O₂, oxygen uptake. Data are reported as Pearson r-value (P-value).

### Exercise Oscillatory Ventilation

Table 4 details the mean (absolute), amplitude, and period for \dot{V}_{E}, V_{T}, P_{ET}CO₂, and \dot{V}O₂ during EOV. As was the case for resting PB, there was no difference between the manual hand score and the sine wave algorithm for mean absolute values of \dot{V}_{E}, V_{T}, P_{ET}CO₂, or \dot{V}O₂. Similarly, there were no differences between the hand score and the peak detection algorithm for amplitude for the same variables. The sine wave fit tended to produce smaller amplitude estimates compared to hand score or the peak detect method, and these differences were significant for all variables except \dot{V}_{E}. The peak detect method produced shorter periods compared to manual (P<0.05 for all variables) and the sine wave fit. As with the resting data, correlation coefficients between peak detect and manual methods (Table 5) appeared to be lower for all variables compared to the correlations between sine wave fit and manual.

TABLE 4
Analysis of Exercise Oscillatory Ventilation across Measurement Techniques.

| | Manual | Peak Detect | Sine Wave | Fourier |
|---|---|---|---|---|
| Mean (Absolute) | | | | |
| \dot{V}_{E} (L*min⁻¹) | 19.1 ± 7.5 | — | 19.1 ± 7.6 | — |
| V_{T} (L) | 0.84 ± 0.37 | — | 0.83 ± .37 | — |
| P_{ET}CO₂ (mmHg) | 31.5 ± 5.2 | — | 31.3 ± 4.9 | — |
| \dot{V}O₂ (L*min⁻¹) | 0.47 ± 0.20 | — | 0.47 ± 0.20 | — |
| Amplitude | | | | |
| \dot{V}_{E} (L*min⁻¹) | 13.0 ± 6.3 | 14.0 ± 6.8 | 11.5 ± 6.5 | — |
| V_{T} (L) | 0.55 ± .37 | 0.62 ± 0.41 | 0.43 ± 0.38 * | — |
| P_{ET}CO₂ (mmHg) | 7.66 ± 3.12 | 8.04 ± 4.27 | 6.21 ± 3.40 * | — |
| \dot{V}O₂ (L*min⁻¹) | 0.34 ± 0.22 | 0.34 ± 0.22 | 0.22 ± 0.13 * | — |
| Period (minutes) | | | | |
| \dot{V}_{E} | 1.06 ± 0.33 | 0.81 ± 0.26 * | 1.06 ± 0.36 | 1.07 ± 0.28 |
| V_{T} | 1.08 ± 0.37 | 0.70 ± 0.23 * | 1.06 ± 0.36 | 1.02 ± 0.28 |
| P_{ET}CO₂ | 1.06 ± 0.38 | 0.78 ± 0.17 * | 1.06 ± 0.36 | 1.02 ± 0.40 |
| \dot{V}O₂ | 1.00 ± 0.27 | 0.66 ± 0.21 * | 1.06 ± 0.36 | 1.03 ± 0.26 |

\dot{V}_{E}, minute ventilation; V_{T}, tidal volume; P_{ET}CO₂, partial pressure of end tidal carbon dioxide; \dot{V}O₂, oxygen uptake. Data are reported as mean ± SD. All comparisons vs. Manual.
* = P < 0.05

TABLE 5
Pearson Correlation Analysis of Exercise Oscillatory Ventilation.

| | Peak Detect | Sine Fit | Fourier |
|---|---|---|---|
| Mean (Absolute) | | | |
| \dot{V}_{E} | — | 0.99 (<0.001) | — |
| V_{T} | — | 0.99 (<0.001) | — |
| P_{ET}CO₂ | — | 0.99 (<0.001) | — |
| \dot{V}O₂ | — | 0.99 (<0.001) | — |
| Amplitude | | | |
| \dot{V}_{E} (L/min) | 0.95 (<0.001) | 0.93 (<0.001) | — |
| V_{T} (L) | 0.89 (<0.001) | 0.96 (<0.001) | — |
| P_{ET}CO₂ (%) | 0.75 (0.008) | 0.59 (0.054) | — |
| \dot{V}O₂ (L/min) | 0.67 (0.023) | 0.70 (0.018) | — |
| Period (minutes) | | | |
| \dot{V}_{E} | 0.76 (0.007) | 0.59 (0.057) | 0.98 (<0.001) |
| V_{T} | 0.51 (0.109) | 0.64 (0.033) | 0.94 (<0.001) |
| P_{ET}CO₂ | 0.59 (0.094) | 0.73 (0.009) | 0.93 (<0.001) |
| \dot{V}O₂ | 0.06 (0.852) | 0.69 (0.018) | 0.89 (<0.001) |

\dot{V}_{E}, minute ventilation; V_{T}, tidal volume; P_{ET}CO₂, partial pressure of end tidal carbon dioxide; \dot{V}O₂, oxygen uptake. Data are reported as Pearson r-value (P-value).

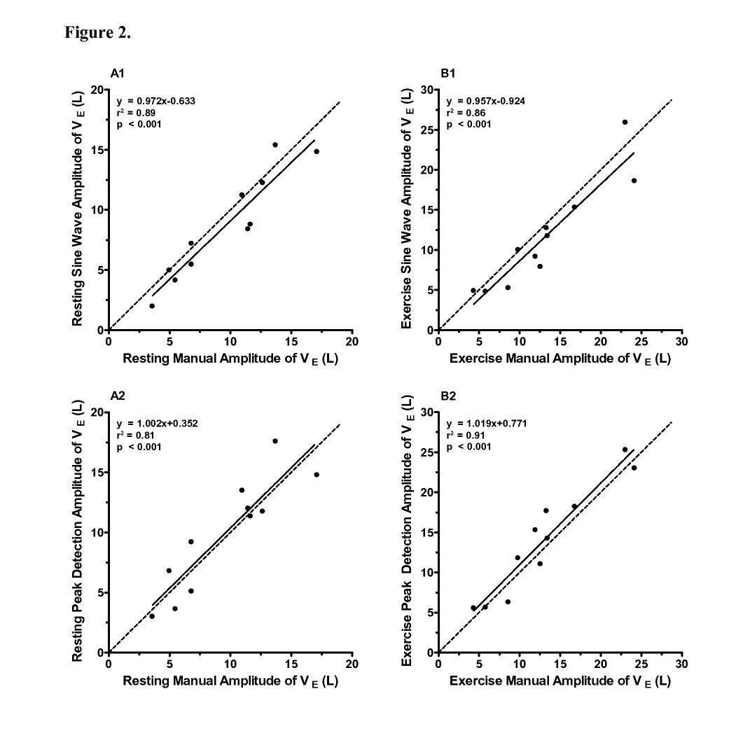

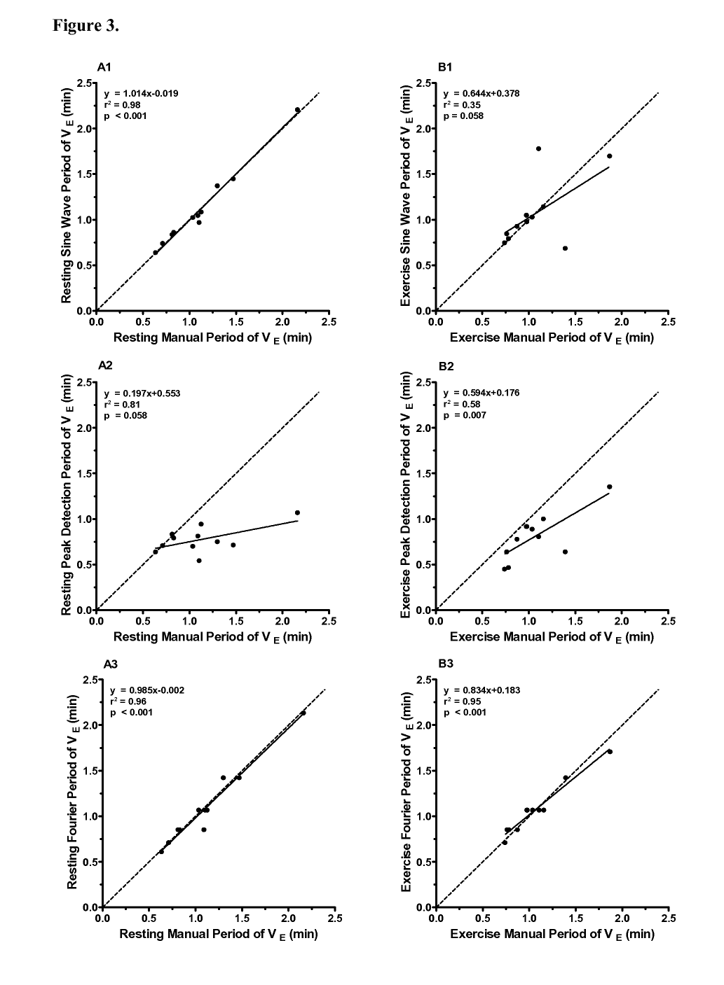

Correlation plots for amplitude of oscillations from the three models were compared to the manual hand score for \dot{V}_{E} at rest and exercise (FIG. 2). In addition, correlation plots for period of oscillations from the three models were compared to manual hand score for \dot{V}_{E} at rest and exercise (FIG. 3). There were good correlations between amplitude and period estimates using the sine wave model, though correlations were slightly lower using the peak detect model. In addition, the Fourier analysis gave very good agreement with manual estimation of cycle period. Correlations of oscillatory periods determined using the peak detection method compared to manual method were much lower.

This work compared three software algorithms for the rapid quantitation of periodic breathing at rest and during exercise against traditional hand scoring in patients with heart failure. The results provided herein demonstrate that the sine wave fitting and Fourier analysis captured the period of oscillations consistently when compared to hand scoring. In contrast, the sine wave fit consistently under-estimated amplitudes. The peak detection algorithm captured amplitudes of oscillation, though largely underestimated the periods. Thus, an optimal combination of fitting involves using the amplitudes from peak detection and periods from either the Fourier or sine wave methods.

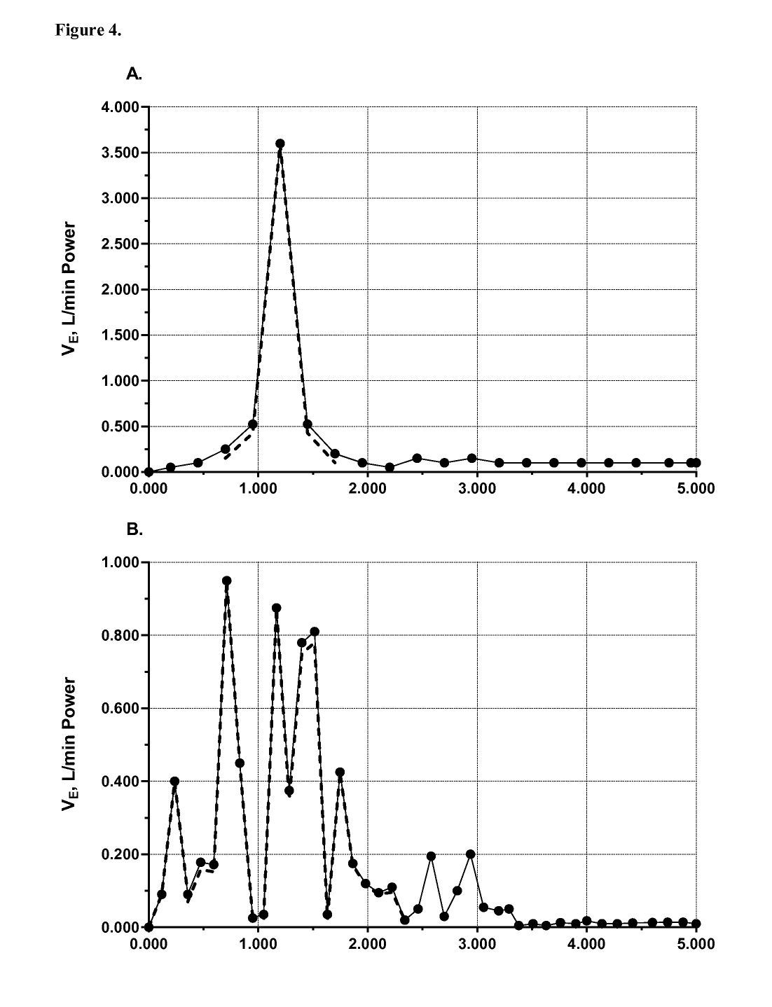

The finding that the peak detection captured amplitudes whereas sine wave fitting underestimated amplitudes is likely a result that reflects the properties of fitting a sine wave to oscillatory ventilation. Due to the underlying principles of a sine wave, the sine wave fitting routine must capture both amplitude and period at a single frequency over oscillations that are not constant in either rate or amplitude. Thus, the best fit sine wave is likely to underestimate amplitude in favor of correctly capturing period. The Fourier analysis can be used to estimate the entire frequency content of the breathing cycles, by quantifying the width of the peaks, and by looking for secondary peaks in the frequency domain. Two examples are shown in FIG. 4. The top panel provides an example that shows a discrete frequency peak, a pattern that would easily be fit with a best-fit sine wave. The bottom panel shows an example with a larger spread in frequency components, and this example proved more difficult to fit with a single-frequency sine wave analysis. These additional measures of frequency components that are not available using manual analysis, peak detection or sine wave fitting can be useful in detailed analyses of oscillatory ventilation. In addition, the ability to clearly define, measure, and quantify disordered breathing, particularly PB at rest and EOV, in a consistent manner, represents a meaningful advance to the HF community.

The techniques and models provided herein can provide the ability to use existing cardiopulmonary exercise test data collected on standard metabolic measurement systems for the quantification of the amplitude and period of oscillations in ventilation and gas exchange measures. The results provided herein demonstrate a close relationship between the peak detection algorithm and the hand score data for the amplitude of oscillations in \dot{V}_{E}, V_{T}, P_{ET}CO₂, and \dot{V}O₂ both at rest and during exercise. In addition, although the sine wave algorithm for measurement of period during exercise was less strong, the Fourier analysis algorithm for measurement of period during both the resting and exercise phases demonstrated strong correlations.

As described herein, in addition to period and amplitude, the sine wave and Fourier analysis fitting routines provided herein can be used to determine phase shifts among variables at rest and during exercise. For example, it was found that P_{ET}CO₂ oscillations consistently lagged the oscillations in \dot{V}_{E} by an average of 151 degrees, though both \dot{V}O₂ and \dot{V}CO₂ were nearly in phase with \dot{V}_{E}. This information can be used to examine basic mechanisms as well as the impact of treatment on oscillatory breathing.

In summary, this study provides three software algorithms for the rapid quantitation of periodic breathing at rest and during exercise. A best-fit sine-wave model and Fourier analysis matched oscillatory periods determined by hand scoring, though the sine wave fit underestimated magnitudes. A peak detection algorithm captured amplitudes of oscillation, though largely underestimated the periods. Output of the sine wave fit and Fourier analyses included goodness of fit measures and can quantify phase shifts among variables, results that can be used in additional studies of phase relationships among oscillatory breathing patterns.

FIG. 5 is a block diagram of computing devices 500, 550 that may be used to implement the systems and methods described in this document, as either a client or as a server or plurality of servers. Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally computing device 500 or 550 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses may be used, as appropriate, along with multiple memories and types of memory. Also, multiple computing devices 500 may be connected, with each device providing portions of the necessary operations (e.g., as a server bank, a group of blade servers, or a multi-processor system).

The memory 504 stores information within the computing device 500. In one implementation, the memory 504 is a volatile memory unit or units. In another implementation, the memory 504 is a non-volatile memory unit or units. The memory 504 may also be another form of computer-readable medium, such as a magnetic or optical disk.

The storage device 506 is capable of providing mass storage for the computing device 500. In one implementation, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 504, the storage device 506, or memory on processor 502.

The high speed controller 508 manages bandwidth-intensive operations for the computing device 500, while the low speed controller 512 manages lower bandwidth-intensive operations. Such allocation of functions is exemplary only. In one implementation, the high-speed controller 508 is coupled to memory 504, display 516 (e.g., through a graphics processor or accelerator), and to high-speed expansion ports 510, which may accept various expansion cards (not shown). In the implementation, low-speed controller 512 is coupled to storage device 506 and low-speed expansion port 514. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. It may also be implemented as part of a rack server system 524. In addition, it may be implemented in a personal computer such as a laptop computer 522. Alternatively, components from computing device 500 may be combined with other components in a mobile device (not shown), such as device 550. Each of such devices may contain one or more of computing device 500, 550, and an entire system may be made up of multiple computing devices 500, 550 communicating with each other.

Computing device 550 includes a processor 552, memory 564, an input/output device such as a display 554, a communication interface 566, and a transceiver 568, among other components. The device 550 may also be provided with a storage device, such as a microdrive or other device, to provide additional storage. Each of the components 550, 552, 564, 554, 566, and 568, are interconnected using various buses, and several of the components may be mounted on a common motherboard or in other manners as appropriate.

The processor 552 can execute instructions within the computing device 550, including instructions stored in the memory 564. The processor may be implemented as a chipset of chips that include separate and multiple analog and digital processors. Additionally, the processor may be implemented using any of a number of architectures. For example, the processor 552 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 550, such as control of user interfaces, applications run by device 550, and wireless communication by device 550.

Processor 552 may communicate with a user through control interface 558 and display interface 556 coupled to a display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may be provide in communication with processor 552, so as to enable near area communication of device 550 with other devices. External interface 562 may provide, for example, for wired communication in some implementations, or for wireless communication in other implementations, and multiple interfaces may also be used.

The memory 564 stores information within the computing device 550. The memory 564 can be implemented as one or more of a computer-readable medium or media, a volatile memory unit or units, or a non-volatile memory unit or units. Expansion memory 574 may also be provided and connected to device 550 through expansion interface 572, which may include, for example, a SIMM (Single In Line Memory Module) card interface. Such expansion memory 574 may provide extra storage space for device 550, or may also store applications or other information for device 550. Specifically, expansion memory 574 may include instructions to carry out or supplement the processes described above, and may include secure information also. Thus, for example, expansion memory 574 may be provide as a security module for device 550, and may be programmed with instructions that permit secure use of device 550. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that may be received, for example, over transceiver 568 or external interface 562.

Device 550 may communicate wirelessly through communication interface 566, which may include digital signal processing circuitry where necessary. Communication interface 566 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 568. In addition, short-range communication may occur, such as using a Bluetooth, WiFi, or other such transceiver (not shown). In addition, GPS (Global Positioning System) receiver module 570 may provide additional navigation- and location-related wireless data to device 550, which may be used as appropriate by applications running on device 550.

Device 550 may also communicate audibly using audio codec 560, which may receive spoken information from a user and convert it to usable digital information. Audio codec 560 may likewise generate audible sound for a user, such as through a speaker, e.g., in a handset of device 550. Such sound may include sound from voice telephone calls, may include recorded sound (e.g., voice messages, music files, etc.) and may also include sound generated by applications operating on device 550.

The computing device 550 may be implemented in a number of different forms, as shown in the figure. For example, it may be implemented as a cellular telephone 580. It may also be implemented as part of a smartphone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or

LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A computer-implemented method, wherein said method comprises:
(a) accessing, by a computer system, cardiopulmonary data for a patient;
(b) identifying, using an oscillation period operation, periods of oscillation from the cardiopulmonary data for the patient;
(c) identifying, using peak detection operation, amplitudes of oscillation from the cardiopulmonary data for the patient;
(d) determining, by the computer system, one or more breathing patterns for the patient based on (i) the identified periods of oscillation and (ii) the identified amplitudes of oscillation; and
(e) outputting, by the computer system, information that identifies the one or more breathing patterns.

2. The computer-implemented method of claim 1, wherein the oscillation period operation comprises a sine wave fitting operation that identifies the periods of oscillation.

3. The computer-implemented method of claim 1, wherein the oscillation period operation comprises Fourier analysis operation that identifies the periods of oscillation.

4. The computer-implemented method of claim 1, wherein the cardiopulmonary data for the patient corresponds to measurements that were taken over a period of time while the patient was exercising.

5. The computer-implemented method of claim 1, wherein the cardiopulmonary data for the patient corresponds to measurements that were taken over a period of time while the patient was resting.

6. The computer-implemented method of claim 1, wherein the cardiopulmonary data comprises spirometry and gas exchange values for the patient.

7. The computer-implemented method of claim 1, wherein the one or more breathing patterns comprise cyclic patterns in breath-by-breath ventilation and gas exchange.

8. The computer-implemented method of claim 1, wherein the determining comprises curve fitting the cardiopulmonary data for the patient by combining the periods of oscillation that are identified using the oscillation period operation with the amplitudes of oscillation that are identified using the peak detection operation.

9. The computer-implemented method of claim 1, wherein the peak detection operation comprises:
(a) for each data point in the cardiopulmonary data, generating a list that includes at least a threshold number of adjacent data points that occur earlier and later in time within the cardiopulmonary data;
(b) sorting the generated list;
(c) discarding data points in the generated list have the greatest and lowest values to generate a resulting list;
(d) identifying local minima and maxima from the resulting list;
(e) determining time differences and magnitude differences between the local minima and maxima; and
(f) averaging the time differences and the magnitude differences to determine magnitude and period values from the cardiopulmonary data.
